(19) United States
(12) Patent Application Publication
(10) Pub. No.: US 2008/0167525 A1
(43) Pub. Date: Jul. 10, 2008

(54) Magnetically Propelled Capsule Endoscopy

(76) Inventor: Glenn Mark Wakefield, Tempe, AZ (US)

Correspondence Address: Glenn Wakefield, 1416 East Casmen Street, Tempe, AZ, 85283, US

(21) Appl. No.: 10/570,022

(22) PCT Filed: Mar. 29, 2004
(86) PCT No.: PCT/US2004/009471

§ 371 Date: Feb. 24, 2006

Related U.S. Application Data
(62) Division of application No. 10/406,336, filed on Apr. 3, 2003, now abandoned.

(51) Int. Cl. A61B 1/00

(52) U.S. Cl. 600/118

(57) ABSTRACT

Magnetically propelled capsule endoscopy provides for the medical examination and treatment of the gastrointestinal tract, reproductive tract, trachea, lungs, vascular system or any accessible body cavity. The capsule will be steered throughout the system by an external magnetic field. This instrument will provide for various real time imaging modalities, for physical property measurements and for tissue biopsy and tissue excision. This instrument allows any appropriate medical or non medical system to undergo the appropriate procedure.

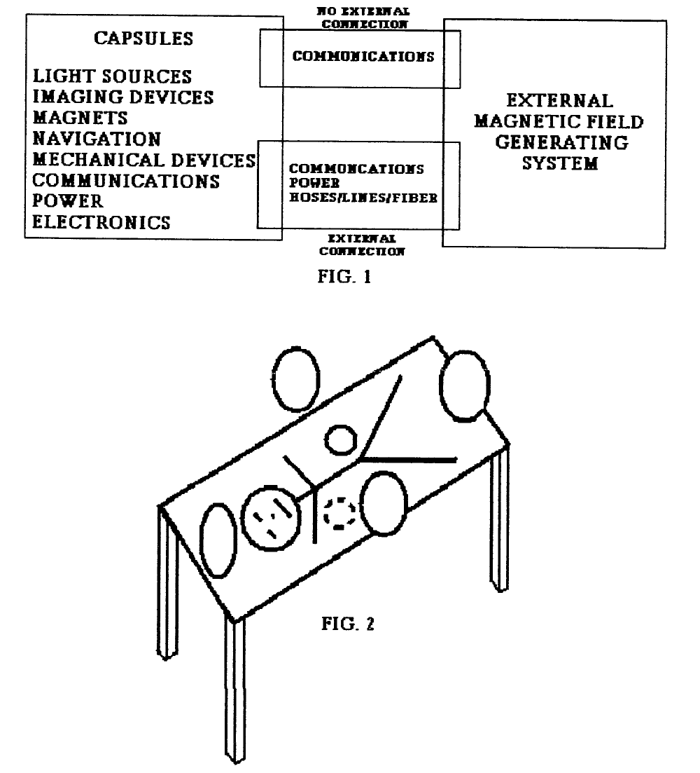

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

This patent application may be applied to the field of medical imaging, tissue biopsy/excision and system imaging (i.e. non medical).

The digestive tract may be examined by the upper gastrointestinal endoscope, the lower gastrointestinal endoscope and the capsule endoscope. The upper gastrointestinal endoscope and the lower gastrointestinal endoscope require an anesthetic and are limited by the inability to examine the small intestine. The capsule endoscope does not permit real time imaging and precludes tissue biopsy/excision. All three methods provide a limited range of viewing/access angles.

### BRIEF SUMMARY OF THE INVENTION

Magnetically propelled capsule endoscopy provides for the medical examination of the gastrointestinal tract, reproductive tract, trachea/lungs, vascular system or any accessible body cavity. The capsule will be steered throughout the system by an external magnetic field. This instrument will provide for various real time imaging modalities, for physical property measurements and for tissue biopsy/excision. This instrument allows any appropriate system (i.e. non medical) to undergo analysis.

### BRIEF DESCRIPTION OF THE SEVERAL VIEWS OF THE DRAWING

Not Applicable

### DETAILED DESCRIPTION OF THE INVENTION

This instrument may be used for the purpose of performing a medical examination of the digestive tract, reproductive tract, trachea/lungs, vascular system or any accessible body cavity. Other non medical systems may be effectively analyzed by the instrument. A capsule will be inserted into the appropriate location. Contained within the capsule may be the following basic components:

(#1 possibility)—magnets or magnetizable materials for movement and direction control; magnetic sensors; light sources; imaging lenses; transmitter and receiver for communication; magnetic pickup for power generation;

gyroscope/accelerometer for location information; mechanical equipment for tissue biopsy/excision or other procedures; power source; electronics;

(#2 possibility)—magnets or magnetizable materials for movement and direction control; magnetic sensors; light sources, imaging lenses, power lines, communication lines, injection hoses, suction hoses and other lines/hoses extending out the back of the capsule and connecting to the external control device; gyroscope/accelerometer for location information; mechanical equipment for tissue biopsy/excision or other procedures; power source; electronics.

The choice of no physical connection (#1 possibility) or physical connection (#2 possibility), capsule size and capabilities will vary depending on the design/application. Many other components as described below may be designed into these two basic configurations.

The magnets or magnetizable material contained within the capsule will be bathed in an external magnetic field. The external magnetic field is created by any number of field generating structures with the arbitrary positioning of each structure. The number and configuration of the structures will depend on the design parameters. For explanation purposes, one structure will be centrally located on each face of an imaginary cube. Current will run through each of the six structures which essentially allows oppositely positioned pairs of structures to control each spatial dimension. The configuration of the instrument will be such that the patient's body passes through diagonally opposite edges of the imaginary cube. The capsule will be moved by appropriately changing the current distribution within each structure and consequently the magnetic field. As necessary, the actual structures and/or patient will be moved in conjunction with the changing current to allow the capsule to be moved throughout the body. This will also minimize the volume and strength of the magnetic field required to examine the patient. When there is no physical connection, power may be transferred to the capsule to recharge the power source through the use of the field generating structures. By holding the capsule stationary with some of the structures, the other structures may be used to rotate the external magnetic field to operate a small generator within the capsule to recharge the power source. The fundamental motion of the generator may be rotary, curvilinear or linear. The medical practitioner, using visual feedback, will guide the capsule throughout the body part under examination. The magnetic field strength will be adjusted appropriately for direction change, curvilinear movement or special positioning for tissue biopsy/excision or other procedures. The programming of a predetermined movement pattern may be viable if an appropriate object (i.e. non medical) is being analyzed by the instrument

The capsule will be able to measure the temperature, pH, substance concentration, pressure, strain, force, magnetic field, electric field and other physical quantities. The capsule will have the ability to detect and produce sound waves, to detect and produce electromagnetic waves (i.e. visible, infrared), to detect and produce elementary/nuclear particles and to examine by other modalities. The gyroscope/accelerometer will provide the ability to map in real time the positional progress of the capsule. A positional map will be created in real time of the entire procedure, areas of interest may be marked, and visual images or other data will be generated corresponding to each positional location of the capsule. The data may be analyzed in real time to produce a virtual three dimensional image of the completed portion of the exam. This will allow the doctor to quickly review the entire exam before removing the capsule. All of this data may be appropriately stored for future reference.

I claim:

1. (canceled)

2. (canceled)

3. (canceled)

4. (canceled)

5. (canceled)

6. (canceled)

7. (canceled)

8. (canceled)

9. (canceled)

10. (canceled)

12. Referring to claim 11, the system allows for a medical examination of the digestive tract, reproductive tract, trachea, lungs, vascular system or any accessible body cavity.

13. Referring to claim 11, the capsule with an external physical connection may have light sources, imaging lenses, power lines, communication lines, injection hoses, suction hoses and other lines and hoses extending from the capsule and connecting to an external control instrument.

14. Referring to claim 11, the capsule without an external physical connection may have light sources, imaging lenses, cameras, communications and a power source.

15. Referring to claim 11, the capsule with or without an external physical connection may have electronic control, magnetic sensors, the ability to operate under magnetic conditions, mechanical equipment for tissue biopsy and tissue excision or other procedures and the programming of a predetermined movement pattern.

16. Referring to claim 11, by holding the capsule stationary with some of the field generating structures, the other field generating structures may be used to manipulate the external magnetic field to operate a small generator (whose motion may be rotary, curvilinear or linear) within the capsule to recharge the power source.

17. Referring to claim 11, the instrument allows for real time display of information, virtual three dimensional image of the completed portion of the exam, real time mapping of the capsule's trajectory via the gyroscope and accelerometer, marking points of interest and storage of data from all sensors and equipment along the entire trajectory.

18. Referring to claim 11, the capsule allows the measurement of temperature, pH, substance concentration, pressure, strain, force, magnetic field, electric field and other physical quantities.

19. Referring to claim 11, the capsule allows the detection and production of sound waves, the detection and production of electromagnetic waves, the detection and production of elementary and nuclear particles and the examination by other modalities.

20. Referring to claim 11, the externally generated magnetic field for controlling the capsules may be created by:
appropriately placing six field generating structures on the six faces of an imaginary cube;
have current running through each of the six field generating structures which effectively allows a pair of field generating structures to control each spatial dimension;
placing the patient undergoing the medical examination inside the imaginary cube;
moving the capsule by appropriately changing the field generating structure current and position and the patient's position.
